(19) United States
(12) Patent Application Publication
(10) Pub. No.: US 2023/0357700 A1
(43) Pub. Date: Nov. 9, 2023

(54) Sampling Device And Cell Culturing System

(71) Applicant: Terumo Kabushiki Kaisha, Tokyo (JP)

(72) Inventor: Masatsugu IGARASHI, Kanagawa (JP)

(21) Appl. No.: 18/224,415

(22) Filed: Jul. 20, 2023

Related U.S. Application Data
(63) Continuation of application No. PCT/JP2022/008732, filed on Mar. 2, 2022.

(30) Foreign Application Priority Data
Mar. 3, 2021 (JP) 2021-033639

(51) Int. Cl. C12M 1/34; C12M 1/00

(52) U.S. Cl. CPC C12M 41/34; C12M 29/00

(57) ABSTRACT

A cell culturing system includes a sampling channel, a detection unit, and a first measuring instrument that measures a gas component contained in a sample by means of the detection unit while the sample flows. The sampling device also includes a standard solution storage unit capable of supplying a standard solution to the detection unit through the sampling channel, and a gas supply device that supplies a gas having a predetermined component amount to the standard solution in the standard solution storage unit. The first measuring instrument performs calibration by measuring the standard solution mixed with the gas having the predetermined component amount.

### CROSS-REFERENCE TO RELATED APPLICATIONS

The present application is a continuation application of the International Patent Application No. PCT/JP2022/008732 filed on Mar. 2, 2022, which designated the U.S. and claims the benefit of priority from Japanese Patent Application No. JP2021-033639 filed on Mar. 3, 2021. The entire disclosures of the above-identified applications are incorporated herein by reference.

### FIELD

The present disclosure relates to a sampling device for collecting a liquid sample from a cell culturing device and to a cell culturing system that includes the sampling deice and the cell culturing device.

### BACKGROUND

A sampling device for collecting a liquid sample from a culturing device includes a sampling channel and a pump that is configured to draw a sample from a sample introduction channel that connects the sampling channel to the cell culturing device. The sampling channel also includes a detection unit. The detection unit can detect components such as oxygen (O₂) and/or carbon dioxide (CO₂) in the sample and the amounts (concentrations) of the components.

A measuring instrument can perform measurement for the detection unit and needs to be calibrated at an appropriate timing. The calibration of the measuring instrument may be performed in such a way that a reference measurement value of the measuring instrument is set by the measuring instrument measuring a standard solution having a prescribed O₂ concentration and/or CO₂ concentration. The sampling device may include a calibration device for bubbling (mixing) gas having a prescribed O₂ concentration and/or CO₂ concentration into the standard solution.

Such calibration devices, however, can lead to an increase in size of the sampling device. Further, a user needs to set the measuring instrument in the calibration device for calibration, which can increase a work burden of the user.

Accordingly, there is a need for a sampling device and/or a cell culturing system capable of more efficiently calibrating a measuring instrument.

### SUMMARY

In at least one example embodiment, the present disclosure provides a sampling device for collecting a liquid sample from a cell culturing device that cultures a cell. The sampling device may include a sampling channel through which the sample flows, a detection unit provided in the sampling channel so as to come into contact with the sample, and a measuring instrument that measures at least a gas component contained in the sample by means of the detection unit while the sample flows. The sampling device may also include a standard solution storage unit capable of supplying a standard solution to the detection unit through the sampling channel and a gas supply unit that is configured to supply a gas having a predetermined component amount to the standard solution in the standard solution storage unit. The measuring instrument may perform calibration by measuring the standard solution mixed with the gas.

In at least one example embodiment, the present disclosure provides a cell culturing system that includes a culturing unit for culturing a cell a sampling channel through which a liquid collected from the culturing unit flows, a detection unit provided in the sampling channel so as to come into contact with the sample, and a measuring instrument that is configured to measure at least a gas component contained in the sample by means of the detection unit while the sample flows. The cell culturing system may also include a standard solution storage unit capable of supplying a standard solution to the detection unit through the sampling channel and a gas supply unit that is configured to supply a gas having a predetermined component amount to the standard solution in the standard solution storage unit. The measuring instrument may perform calibration by measuring the standard solution mixed with the gas having the predetermined component amount.

The sampling device and the cell culturing system of the present disclosure may enable easy calibration of the measuring instrument and may also allow the device and system to have a smaller, more manageable size and may improve efficiency and usability.

### BRIEF DESCRIPTION OF DRAWINGS

FIG. 1 is a perspective view of a cell culturing system including a sampling device in accordance with at least one example embodiment of the present disclosure.

FIG. 2 is a schematic illustrating a channel for a culture medium in the cell culturing system illustrated in FIG. 1 in accordance with at least one example embodiment of the present disclosure.

FIG. 3 is a schematic illustrating a channel of the sampling device as illustrated in FIG. 1 in accordance with at least one example embodiment of the present disclosure.

FIG. 4 is a perspective view illustrating a first sensor unit and a second sensor unit of the sampling device illustrated in FIG. 3 in accordance with at least one example embodiment of the present disclosure.

FIG. 5 is a schematic illustrating an example standard solution storage unit and an example gas supply device of the sampling device illustrated in FIG. 1 in accordance with at least one example embodiment of the present disclosure.

FIG. 6A is a schematic illustrating an example gas supply device in accordance with at least one example embodiment of the present disclosure.

FIG. 6B is a schematic illustrating another example gas supply device in accordance with at least one example embodiment of the present disclosure.

FIG. 7 is a flowchart illustrating a sampling method as performed by the sampling device illustrated in FIG. 1 in accordance with at least one example embodiment of the present disclosure.

FIG. 8 is a schematic illustrating operations in the priming step and the cleaning step of the sampling method illustrated in FIG. 7 in accordance with at least one example embodiment of the present disclosure.

FIG. 9 is a schematic illustrating an operation in the sampling step of the sampling method illustrated in FIG. 7 in accordance with at least one example embodiment of the present disclosure.

FIG. 10 is a flowchart illustrating a flowchart of processing in the calibration step of the sampling method illustrated in FIG. 7 in accordance with at least one example embodiment of the present disclosure.

FIG. 11A is a schematic illustrating an operation of supplying the first gas during the calibration step as illustrated in FIG. 10 in accordance with at least one example embodiment of the present disclosure.

FIG. 11B is a schematic illustrating a stand-by state in which the operation of the gas supply device is stopped during the calibration step as illustrated in FIG. 10 in accordance with at last one example embodiment of the present disclosure.

FIG. 11C is a schematic illustrating an operation of supplying the second gas during the calibration step as illustrated in FIG. 10 in accordance with at last one example embodiment of the present disclosure.

FIG. 12 is a schematic illustrating an operation during the calibration step as illustrated in FIG. 10 in accordance with at last one example embodiment of the present disclosure.

FIG. 13 is a schematic illustrating another example gas supply device in accordance with at least one example embodiment of the present disclosure.

### DETAILED DESCRIPTION

Example embodiments of the present disclosure will now be described in detail with reference to the drawings.

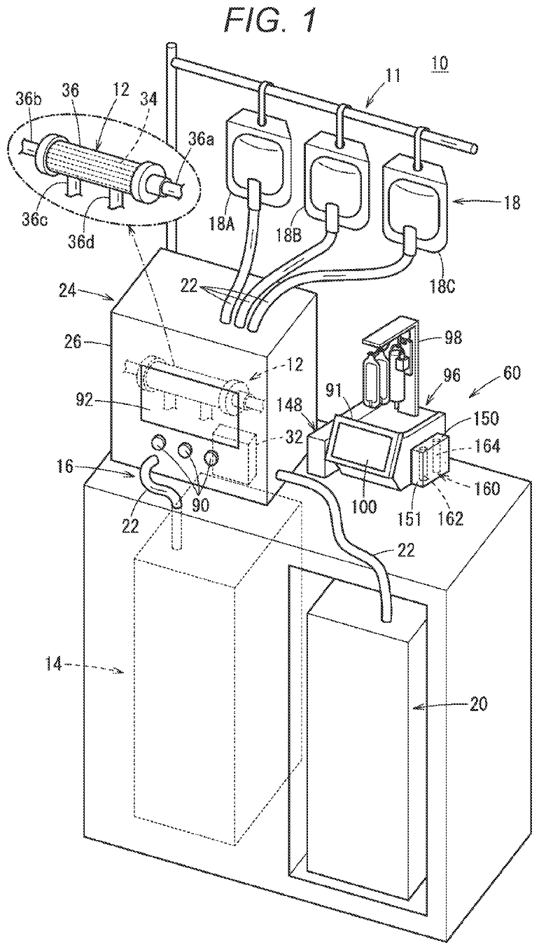

A cell culturing system 10 for culturing biological cells in regenerative medicine is illustrated in FIG. 1. A sampling device 60 may be used with the cell culturing system 10. The sampling device 60 may be configured to sample a culture medium during culture of the cells by the cell culturing system 10 and to measure the state of the culture medium. For example, the cell culturing system 10 may continue cell culture for a long period of time by discharging lactic acid, carbon dioxide, and/or the like (including, for example, unused medium and oxygen) as generated during cell culture from a reactor 12, which may be a cell culture vessel, while supplying a culture medium or oxygen to the reactor 12.

The biological cells are not particularly limited. In at least one example embodiment, the biological cells may include cells contained in blood (e.g., T cells and/or the like) and/or stem cells (e.g., ES cells, iPS cells, mesenchymal stem cells, and/or the like). Any appropriate culture medium may be selected for use with the selected biological cells. For example, the culture medium may include a basic solution that includes amino acids, vitamins, serum, and/or the like. The basic solution may include, for example, a balanced salt solution (BSS).

The cell culturing system 10 may include a culturing device 11 (also referred to as a culturing unit) in which a reactor 12 is set and cells are actually cultured and a sampling device 60 (also referred to as a sampling unit) that collects a liquid sample from the culturing device 11 during the culture. Although only one reactor 12 is illustrated in FIG. 1, it should be appreciated that in other embodiments, the culturing device 11 may include a plurality of reactors 12. Although only one culturing device 11 is illustrated in FIG. 1, it should be appreciated that in other embodiments, the cell culturing system 10 may include a plurality of culturing devices 11 connected to one sampling device 60. Although the cell culturing system 10 as illustrated in FIG. 1 includes the culturing unit and the sampling unit as separate units, components, parts, or pieces, it should be appreciated that in other embodiments, the culturing unit and the sampling unit may be provided as an integrated (unified) unit or component or part or piece.

The culturing device 11 may include a culture medium reservoir 14 configured to store a culture medium, a flow channel 16 provided between the reactor 12 and the culture medium reservoir 14, a plurality of medical bags 18 connected to the flow channel 16, and/or a waste liquid unit 20 configured to store a liquid discharged through the flow channel 16.

The culture medium reservoir 14 may include a hard tank that is configured to store a comparatively large amount of culture medium. The flow channel 16 may include multiple tubes 22. The multiple tubes 22 may be connected, respectively, to the reactor 12, the culture medium reservoir 14, the plurality of medical bags 18, and/or the waste liquid unit 20.

The plurality of medical bags 18 may include a cell solution bag 18A configured to store a liquid (including cells (e.g., cell solution), a cleaning solution bag 18B configured to store a cleaning solution, a stripping solution bag 18C configured to store a stripping solution, and/or a collection bag (not illustrated) configured to collect cultured cells. The cleaning solution may include a liquid used at the time of priming of the reactor 12 and/or the flow channel 16. The cleaning solution may include a buffer solution and/or a physiological saline solution. The buffer solution may include, for example, phosphate buffer salts (PBS) and/or tris-buffered saline (TBS). The stripping solution may include a liquid for stripping the cells cultured by a culture treatment. For example, the stripping solution may include trypsin and/or EDTA.

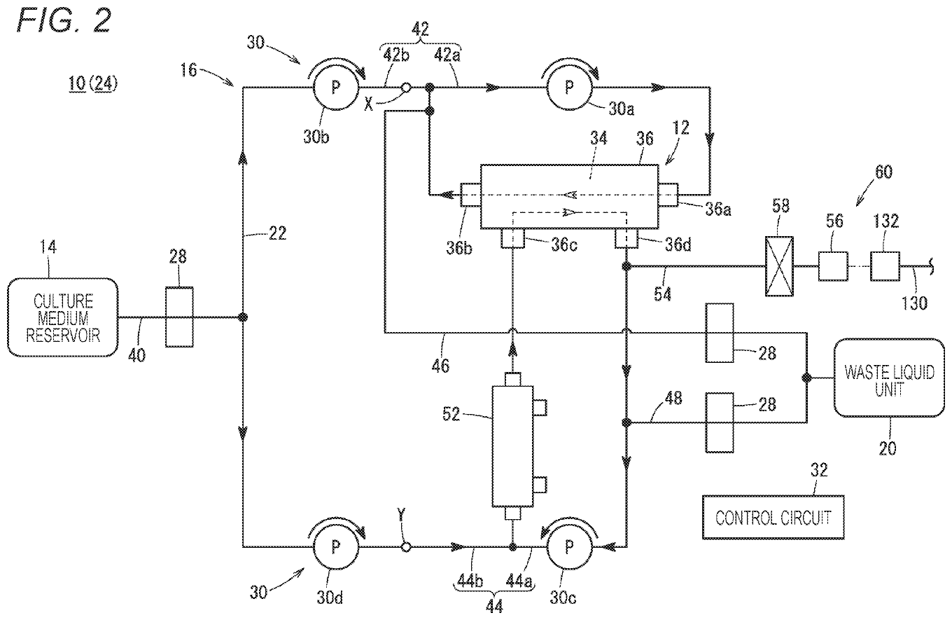

When the cell culturing system 10 is constructed, the flow channel 16 may be set so as to pass through a flow path control mechanism 24 of the culturing device 11. The flow path control mechanism 24 may include a housing 26 that is configured to house a part of the flow channel 16. As illustrated in FIG. 2, the flow path control mechanism 24 may include a plurality of clamps 28 that are configured to open and close a predetermined tube 22, a pump 30 that is configured to allow a liquid in the tube 22 to flow, and/or a control circuit 32 that is configured to control operations of the clamps 28 and/or the pump 30.

The reactor 12 may be disposed in the housing 26 of the flow path control mechanism 24. The reactor 12 may include a plurality of hollow fibers 34 (e.g., 10,000 or more) and a case 36 that accommodates the plurality of hollow fibers 34. Each of the hollow fibers 34 may include a lumen (not illustrated), and cells may be seeded on an inner peripheral surface defining the lumen. Each of the hollow fibers 34 may include a plurality of pores (not illustrated) allowing communication between the outside of the hollow fibers and the lumen. For example, each pore may be sized to transmit a solution or a low-molecular-weight substance without transmitting cells or proteins. A culture medium or the like may be supplied to the cells seeded on the inner peripheral surface of the hollow fiber 34 through the lumen and/or the pores. The configuration in which the liquid mainly flows through the lumen of the hollow fiber 34 may be referred to as intra capillary (IC), and the configuration in which the liquid mainly flows through the outer side of the hollow fiber 34 may be referred to as extra capillary (EC).

Each of the cases 36 may include a first IC terminal 36a and/or a second IC terminal 36b that communicate with the lumens of the hollow fibers 34. Each of the cases 36 may also include a first EC terminal 36c and/or a second EC terminal 36d that communicate with a space outside the hollow fibers 34 in the case 36. The tube 22 may be connected to each terminal.

As illustrated in FIG. 2, the flow channel 16 may include a medium delivery route 40 connected to the culture medium reservoir 14 and an IC route 42 (internal route) and an EC route 44 (external route) which are branched from the medium delivery route 40. The IC route 42 may include a channel for supplying a liquid to the lumen of the hollow fibers 34. The EC route 44 may include a path for supplying liquid into the case 36 outside the hollow fibers 34.

The IC route 42 may include an IC circulation circuit 42a capable of circulating liquid with the reactor 12 and/or an IC supply circuit 42b through which liquid can flow from the culture medium delivery route 40 to the IC circulation circuit 42a. The IC circulation circuit 42a may be connected to the first IC terminal 36a and the second IC terminal 36b of the reactor 12 and may include an IC circulation pump 30a that allows liquid to flow through the lumen of the hollow fibers 34. An IC waste liquid circuit 46 that discharges a culture medium to the waste liquid unit 20 may be connected to the IC circulation circuit 42a on the downstream side of the reactor 12. The IC supply circuit 42b may include an IC supply pump 30b configured to allow liquid to flow from the culture medium delivery route 40 to the IC circulation circuit 42a.

The EC route 44 may include an EC circulation circuit 44a capable of circulating liquid with the reactor 12 and/or an EC supply circuit 44b through which liquid can flow from the culture medium delivery route 40 to the EC circulation circuit 44a. The EC circulation circuit 44a may be connected to the first EC terminal 36c and the second EC terminal 36d of the reactor 12 and may include an EC circulation pump 30c that circulates liquid on the outside of the hollow fibers 34. A gas exchanger 52 may be provided upstream of the reactor 12 in the EC circulation circuit 44a. The gas exchanger 52 may be configured to discharge carbon dioxide mixed in the culture medium and to mix a predetermined gas component (for example, nitrogen N₂: 75%, oxygen O₂: 20%, and/or carbon dioxide CO₂: 5%) with the culture medium. An EC waste liquid circuit 48 that discharges a culture medium to the waste liquid unit 20 may be connected to the EC circulation circuit 44a downstream of the reactor 12. The EC supply circuit 44b may be provided with an EC supply pump 30d for allowing liquid to flow from the culture medium delivery route 40 to the EC circulation circuit 44a.

A plurality of medical bags 18 (cell solution bag 18A, cleaning solution bag 18B, and/or stripping solution bag 18C) may be connected to the IC supply circuit 42b upstream of the IC supply pump 30b or the EC supply circuit 44b upstream of the EC supply pump 30d via a plurality of tubes 22 in addition to the culture medium reservoir 14. It should be appreciated that in certain embodiments, the medical bags 18 may be replaced with a collection bag and/or the like using a sterile connecting device that sterilizes and bonds the bag depending on the intended use.

The sampling device 60 may be connected to the EC circulation circuit 44a of the culturing device 11 at a position (between the reactor 12 and the EC waste liquid circuit 48) near the downstream side (second EC terminal 36d) of the reactor 12. One end of a sample outflow channel 54 through which a culture medium as a liquid sample flows out may be connected to the EC circulation circuit 44a. A culturing-device-side connector 56 may be provided at the other end of the sample outflow channel 54. The culturing-device-side connector 56 may be mutually connectable to a sampling-device-side connector 132 of the sampling device 60.

An aseptic filter 58 may be provided at an intermediate position of the sample outflow channel 54. The aseptic filter 58 may help to maintain an aseptic state of the culture medium circulating through the culturing device 11 (EC circulation circuit 44a). In the sampling device 60, the sample outflow channel 54 may be connected to the IC circulation circuit 42a downstream (second IC terminal 36b) of the reactor 12.

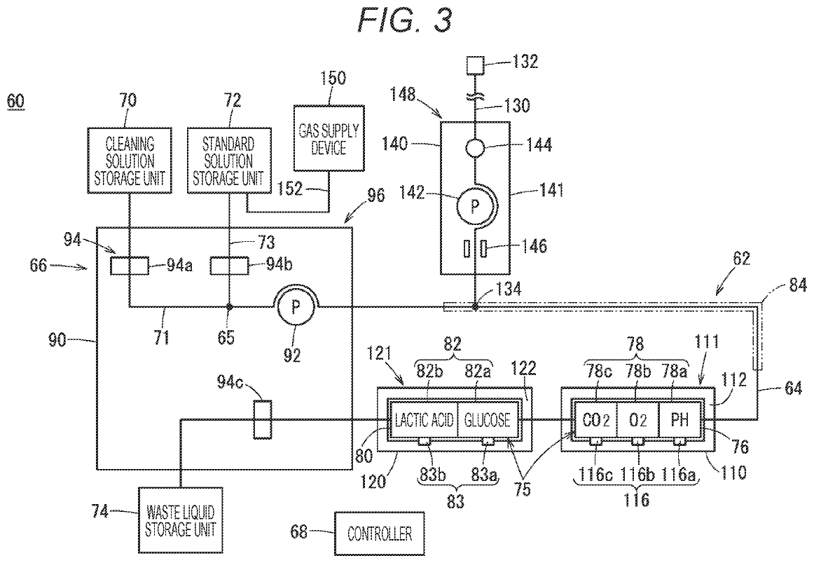

The sampling device 60 is illustrated in FIG. 3. The sampling device 60 may be configured to collect a sample of a culture medium from one or more culturing devices 11 and to detect components contained in the sample and amounts (concentrations) of the components. The sampling device 60 may include a sampling kit 62 having a sampling channel 64 through which a sample is collected, a plurality of mechanisms 66 in which the sampling kit 62 may be detachably set, and/or a controller 68 that is configured to control operations of the plurality of mechanisms 66. In at least one example embodiment, the sampling kit 62 may be disposable product and the plurality of mechanisms 66 may be a reusable product.

In addition to the sampling channel 64, the sampling kit 62 may include a cleaning solution storage unit 70, a standard solution storage unit 72, a waste liquid storage unit 74, and/or a detection unit 75 (including, for example, a first detection unit 76 and a second detection unit 80). The sampling channel 64 may include a flexible tube having an appropriate thickness by which the sample can pass therethrough. The cleaning solution storage unit 70 may be connected to a branch point 65 to which one end of the sampling channel 64 may be connected, for example, via a cleaning solution branch path 71. The standard solution storage unit 72 may also be connected to the branch point 65 via a standard solution branch path 73. The other end of the sampling channel 64 may be connected to the waste liquid storage unit 74.

The cleaning solution storage unit 70 and/or the standard solution storage unit 72 may include a soft resin material that is formed into a bag shape (medical bag). The soft resin material may include, for example, polyvinyl chloride and/or polyolefin. The cleaning solution storage unit 70 and the standard solution storage unit 72 are not particularly limited as long as the storage units are configured to store liquid. The waste liquid storage unit 74 may share a tank with the waste liquid unit 20 of the culturing device 11, but it is not limited thereto, and a medical bag or the like may be applied.

The cleaning solution storage unit 70 may be configured to store a cleaning solution. The cleaning solution is not particularly limited and may include, for example, a buffer solution, a physiological saline solution, and/or the like, such as described in the instance of the cleaning solution bag 18B.

The standard solution storage unit 72 may be configured to store a standard solution. The standard solution may include a liquid for calibrating the first detection unit 76 and/or the second detection unit 80. The standard solution may include a liquid whose pH value, glucose value (glucose concentration), and/or lactic acid value (lactic acid concentration) are set to prescribed values. The standard solution storage unit 72 may be connected to a gas supply device 150 (gas supply unit) and may include an O₂ value (oxygen concentration) and/or a CO₂ value (carbon dioxide concentration) that can be set to prescribed values by the gas supply device 150 supplying O₂ and/or CO₂ which are predetermined gas components. The sampling device 60 may include two or more standard solution storage units 72 that are configured to store standard solutions having different prescribed values and to perform two-point calibration for the first detection unit 76 and/or the second detection unit 80 by supplying two or more types of standard solutions at different timings.

The first detection unit 76 and the second detection unit 80 may be provided in series and separated from each other at an intermediate position of the sampling channel 64. It should be appreciated that although the detection unit 75 as illustrated includes the first detection unit 76 and the second detection unit 80 in other embodiments the detection unit 75 may have a structure in which the first detection unit 76 and the second detection unit 80 are integrated and still other embodiments where the first detection unit 76 and the second detection unit 80 are divided into three or more units.

The first detection unit 76 may include a tubular member having multiple first elements 78 that come in contact with (in liquid contact with) the sample in a flow path in the sampling channel 64. The multiple first elements 78 may include, for example, a pH chip 78a for measuring the pH in the sample, an O₂ chip 78b for measuring the O₂ concentration in the sample, and/or a CO₂ chip 78c for measuring the CO₂ concentration in the sample. The pH chip 78a may be colored by reaction with H⁺ and OH⁻. The O₂ chip 78b may be colored by reaction with O₂. The CO₂ chip 78c may be colored by reaction with CO_{2.}

The second detection unit 80 may include a tubular member having multiple second elements 82 that come in contact with (in liquid contact with) the sample in the flow path in the sampling channel 64 and is provided downstream (waste liquid storage unit 74 side) of the first detection unit 76. For example, the multiple second elements 82 may include biosensors that react an enzyme with a circulating sample and detect a current change or the like. The multiple second elements 82 may include, for example, a glucose chip 82a that measures the glucose concentration in the sample and/or a lactic acid chip 82b that measures the lactic acid concentration in the sample.

The glucose chip 82a may be electrically connected to a glucose terminal 83a protruding to the outside of the tubular member. The lactic acid chip 82b may be electrically connected to a lactic acid terminal 83b protruding to the outside of the tubular member. The glucose terminal 83a and the lactic acid terminal 83b may be preferably integrated as an electrode terminal 83 with an insulating material therebetween.

In addition, the sampling kit 62 may include a connection part 84 to which one or more sample introduction channels 130 can be connected between the branch point 65 of the sampling channel 64 and the first detection unit 76. The connection part 84 may include, for example, a member obtained by integrally molding a plurality of branch ports each having a valve (not illustrated) that closes when the sample introduction channel 130 is not attached and opens as the sample introduction channel 130 is attached (in FIG. 3, the connection part 84 is indicated as a range surrounded by a two-dot chain line for convenience). Alternatively, a port to which the sample introduction channel 130 can be connected with the sampling channel 64 being kept aseptic can be applied to the connection part 84.

A part of the sampling kit 62 may be set in a main mechanism 90 which may be one of the plurality of mechanisms 66 as illustrated in FIG. 3. The main mechanism 90 may also include, in the housing 91 (see FIG. 1), a main-mechanism-side pump 92 and a plurality of clamps 94 that are configured to open and close flow paths in the respective channels (tubes). Although not illustrated, it should be appreciated that in at least one example embodiment, the controller 68 that controls the sampling device 60 may be provided in the main mechanism 90.

The sampling channel 64 extending between the branch point 65 and the connection part 84 may be disposed in the main-mechanism-side pump 92. The main-mechanism-side pump 92 may have a circular wound portion around which the sampling channel 64 can be wound so as to wrap around and may be configured to rotate so as to apply a peristaltic action on the wrapping sampling channel 64 (tube) thereby allowing an internal fluid (liquid, air, etc.) to flow.

The multiple clamps 94 may include a cleaning solution clamp 94a configured to open and close the cleaning solution branch path 71, a standard solution clamp 94b configured to open and close the standard solution branch path 73, and/or a waste liquid clamp 94c configured to open and close the sampling channel 64 between the second detection unit 80 and the waste liquid storage unit 74.

The sampling kit 62 may be set in the main mechanism 90 by which a main unit 96 of the sampling device 60 is constructed. The main unit 96 (main mechanism 90) may include a stand 98 for suspending the cleaning solution storage unit 70 and/or the standard solution storage unit 72 on the top of the housing 91 and a door-like monitor 100 on the front surface of the housing 91.

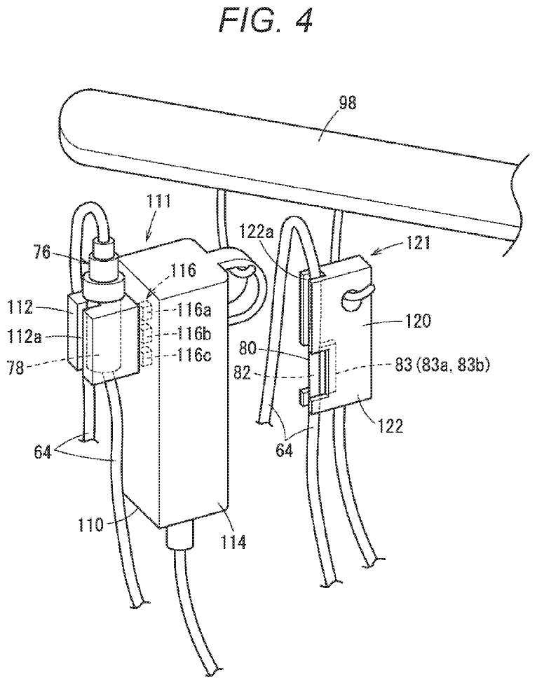

As illustrated in FIGS. 3 and 4, the first detection unit 76 of the sampling kit 62 may be set in a first measuring instrument 110 which is one of the plurality of mechanisms 66. The first measuring instrument 110 may include a holder 112 having a square tube shape and configured to accommodate the plurality of first elements 78. The first measuring instrument 110 may also include a cylindrical measurement body 114 to which the holder 112 may be fixed and which is configured to optically measure the plurality of first elements 78. The holder 112 may be formed to have light shielding properties and may include a recess 112a for housing and holding the first detection unit 76 from the side.

The measurement body 114 may be provided with optical detectors 116 that are arranged to face the plurality of first elements 78 (e.g., pH chip 78a, O₂ chip 78b, and/or CO₂ chip 78c) with the first detection unit 76 being held by the holder 112. The plurality of optical detectors 116 may include a pH detector 116a, an O₂ detector 116b, and/or a CO₂ detector 116c. Under the control of the controller 68, each optical detector 116 may emit measurement light having a wavelength corresponding to the characteristics of the corresponding first element 78 and may be configured to receive excitation light generated from the first element 78 by excitation. Thus, each optical detector 116 may be configured to transmit a detection signal based on the degree of coloration of the corresponding first element 78 to the controller 68.

The second detection unit 80 of the sampling kit 62 may be set in a second measuring instrument 120, which may be one of the plurality of mechanisms 66. The second measuring instrument 120 may include a plate-like case 122 capable of accommodating the plurality of electrode terminals 83 protruding from the plurality of second detection units 80. The case 122 may include a recess 122a for housing and holding the second detection units 80 from the side and a port (not illustrated) into which the electrode terminals 83 may be inserted.

The second measuring instrument 120 may include an enzyme detector (not illustrated) electrically connected to the glucose terminal 83a and/or the lactic acid terminal 83b in a state where the second detection unit 80 is held in the case 122. The enzyme detector may be configured to detect a current value from the glucose chip 82a and/or the lactic acid chip 82b and to transmit a detection signal based on the current value to the controller 68.

In the sampling device 60, a first sensor unit 111 may be constructed by setting the first detection unit 76 in the first measuring instrument 110 and a second sensor unit 121 may be constructed by setting the second detection unit 80 in the second measuring instrument 120. The first sensor unit 111 and the second sensor unit 121 may be provided to be separated from each other, whereby different detection methods are possible and the respective sensor units can be easily set.

In order to introduce a sample to be measured by the first sensor unit 111 and the second sensor unit 121, the sample introduction channel 130 may be connected to the connection part 84 of the sampling kit 62 (sampling channel 64) as illustrated in FIG. 3. Similar to the sampling channel 64, the sample introduction channel 130 may include a flexible tube having an appropriate thickness by which the sample can pass therethrough.

The sample introduction channel 130 may have, at one end, a sampling-device-side connector 132 configured to be connected to the culturing-device-side connector 56 (see also FIG. 2). A plug (not illustrated) attachable to and detachable from the connection part 84 may be provided at the other end of the sample introduction channel 130. A portion where the sample introduction channel 130 is connected to the sampling channel 64 may be referred to as a connection point 134.

A part of the sample introduction channel 130 may be detachably set to an introduction mechanism 140 which is one of the plurality of mechanisms 66. The introduction mechanism 140 may include, within the housing 141, an introduction-mechanism-side pump 142 (second pump), a pressure sensor 144 that is configured to detect the pressure in the flow path of the sample introduction channel 130, and/or a bubble sensor 146 that is configured to detect air bubbles in the flow path of the sample introduction channel 130.

The introduction-mechanism-side pump 142 may include a circular wound portion around which the sample introduction channel 130 can be wound and may be configured to rotate so as to apply a peristaltic action on the wrapping sample introduction channel 130 (tube) thereby allowing an internal fluid (liquid, air, etc.) to flow. The introduction mechanism 140 including the introduction-mechanism-side pump 142 may be set near the connection point 134.

The sample introduction channel 130 may be set in the introduction mechanism 140, by which an introduction unit 148 of the sampling device 60 may be constructed. The introduction unit 148 may be configured such that a part of the sample introduction channel 130, the introduction-mechanism-side pump 142, the pressure sensor 144, and/or the bubble sensor 146 can be integrally handled. The sample introduction channel 130 extending from the introduction unit 148 may be connected to the connection part 84 on the main unit 96.

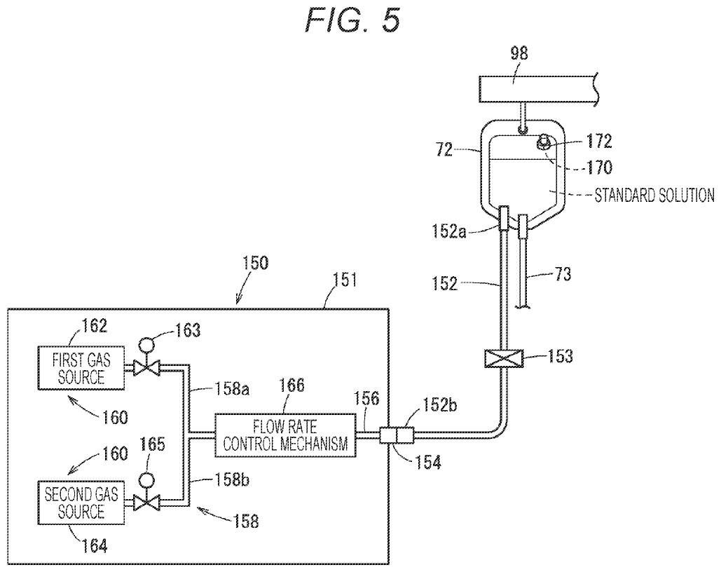

The gas supply device 150 as illustrated in FIG. 5 may be configure to supply gas having a predetermined component amount (O₂ concentration, CO₂ concentration, and/or N₂ concentration) to the standard solution storage unit 72. The gas supply device 150 may be configured to bubbles (mixes) gas into the standard solution in the standard solution storage unit 72 to set the gas (O₂ concentration, CO₂ concentration, and/o N₂ concentration) in the standard solution to a prescribed value.

The gas supply device 150 may be connected to the standard solution storage unit 72 via a gas supply channel 152 which may include a flexible tube. One end of the gas supply channel 152 may be connected in advance to one end side of the standard solution storage unit 72 in the longitudinal direction. Specifically, one end 152a of the gas supply channel 152 may extend parallel to the standard solution branch path 73 vertically below the standard solution storage unit 72 suspended from the stand 98 and may communicate with a space in the standard solution storage unit 72. The one end 152a of the gas supply channel 152 may be preferably provided with a check valve (not illustrated) or the like that is configured to allow the flow of the gas into the standard solution storage unit 72 and to interrupt the flow of the standard solution into the gas supply channel 152.

The other end of the gas supply channel 152 may be provided with a gas-supply-channel-side connector 152b that can be attached to and detached from a gas-supply-device-side connector 154 of the gas supply device 150. In addition, the gas supply channel 152 may include an aseptic filter 153 on the gas supply channel 152 in order to sterilize the standard solution.

The gas supply device 150 may include a housing 151 disposed at a position adjacent to the main mechanism 90. The housing 151 may include a common pipe 156 having one end connected to the gas-supply-device-side connector 154 and a plurality of (two in the present embodiment) branch pipes 158 branching from the other end of the common pipe 156. A gas source 160 may be connected to each of the plurality of branch pipes 158.

Each of the plurality of gas sources 160 may include a tank or the like that stores a gas having a predetermined component amount in a compressed state. One of the plurality of gas sources 160 may be referred to as a first gas source 162 and the other may be referred to as a second gas source 164. The component amount of the first gas of the first gas source 162 and the component amount of the second gas of the second gas source 164 may be different from each other. The component amount of the first gas may be set such that, for example, the N₂ concentration is 75%, the O₂ concentration is 20%, and the CO₂ concentration is 5%. The component amount of the second gas may be set such that, for example, the N₂ concentration is 60%, the O₂ concentration is 30%, and the CO₂ concentration is 10%.

Note that, although the sampling device 60 may include the first gas source 162 and the second gas source 164 in order to perform two-point calibration for calibrating the first sensor unit 111, the gas supply device 150 may only need to include one gas source 160 when one-point calibration is executed. Alternatively, the sampling device 60 may include three or more gas sources 160 in the gas supply device 150 for improving calibration accuracy by the component amounts of three or more different types of gases. The common pipe 156 and the plurality of branch pipes 158 may be appropriately provided according to the number of gas sources 160 installed.

An outlet of the first gas source 162 or the branch pipe 158 (referred to, for example, as a first branch pipe 158a) connected to the first gas source 162 may be provided with a first sealing valve 163 that opens and closes a flow path in the first branch pipe 158a. An outlet of the second gas source 164 or the branch pipe 158 (referred to, for example, as a second branch pipe 158b) connected to the second gas source 164 may be provided with a second sealing valve 165 that opens and closes a flow path in the second branch pipe 158b. The first sealing valve 163 and the second sealing valve 165 may be opened and closed under the control of the controller 68 to selectively allow the first gas and the second gas to flow out to the common pipe 156.

The common pipe 156 may be provided with a flow rate control mechanism 166 for regulating the flow rate of the gas to be supplied to the standard solution storage unit 72. For example, an injector having a valve (not illustrated) whose opening and closing times are controlled may be applied as the flow rate control mechanism 166 and the flow rate of the gas may be regulated by intermittently opening the valve under the control of the controller 68.

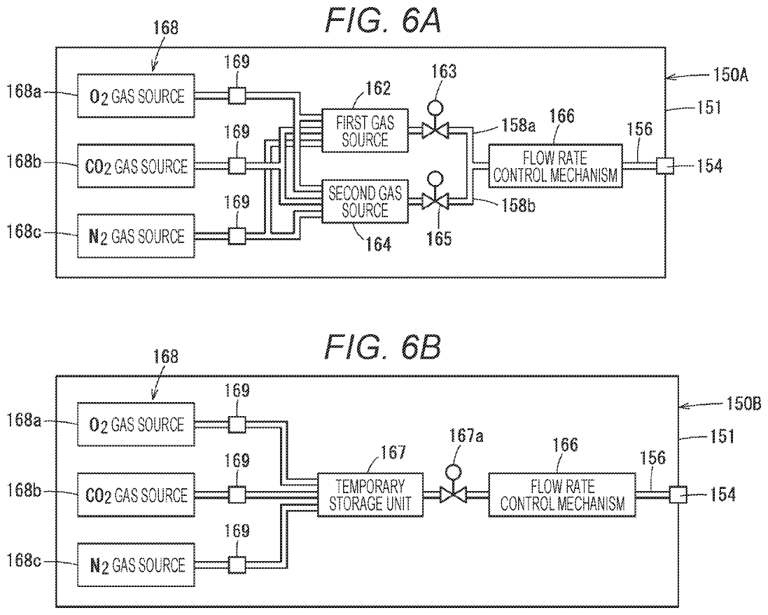

In at least one example embodiment, as illustrated in FIG. 6A, a gas supply device 150A may have a configuration in which each of single-component gas sources 168 (e.g., O₂ gas source 168a, CO₂ gas source 168b, and/or N₂ gas source 168c) may be connected to each of the first gas source 162 and the second gas source 164 and a flow rate control mechanism 169 may be provided downstream of each of the single-component gas sources 168. That is, the gas supply device 150A may be configured to adjust therein the concentrations of O₂, CO₂, and/or N₂ to be supplied and to generate the first gas of the first gas source 162 and the second gas of the second gas source 164. In other example embodiments, as illustrated in FIG. 6B, a gas supply device 150B may include one temporary storage unit 167 to which each of the single-component gas sources 168 (e.g., O₂ gas source 168a, CO₂ gas source 168b, and/or N₂ gas source 168c) may be connected and one sealing valve 167a. That is, the gas supply device 150B may be configured to generate the first gas from the components in the single-component gas sources 168 in the temporary storage unit 167 and to supply the first gas to the standard solution storage unit 72. Then, the gas supply device 150B may be configured to generate the second gas in the same temporary storage unit 167 and to supply the second gas to the standard solution storage unit 72.
the standard solution storage unit 72 may include a discharge port 170 through which the gas in the space can be discharged on the vertically upper side. The gas mixed in the standard solution may be discharged through the discharge port 170 with the lapse of time, whereby the component amount of the gas in the standard solution may vary. The discharge port 170 may be preferably provided with a vent mechanism 172 that is configured to allow permeation of gas and to interrupt permeation of liquid. Thus, the discharge port 170 can prevent the standard solution from flowing out of the standard solution storage unit 72.

The controller 68 (control unit) may include a computer that includes one or more processors (not illustrated), a memory, an input/output interface, and/or an electronic circuit. The controller 68 may be configured to control the entire sampling device 60 when the processor executes the program stored in the memory. At this time, when determining execution of calibration of the first detection unit 76 and the second detection unit 80, the controller 68 may be configured to perform ganged control of the main unit 96 and the gas supply device 150. In at least one example embodiment, the controller 68 may be a control device integrated with the control circuit 32 of the culturing device 11.

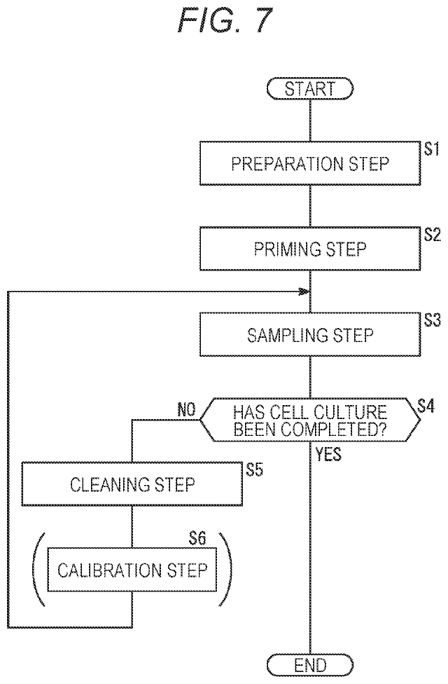

A sampling method performed, for example, using the sampling device 60 is described below in reference to FIG. 7. The sampling method may include, for example, a preparation step, a priming step, a sampling step, a cleaning step, and/or a calibration step, which may be sequentially performed.

In the preparation step (step S1), the user of the cell culturing system 10 may set (attaches) the sampling kit 62 to the main mechanism 90 to form the main unit 96 as illustrated in FIGS. 3 to 5. Thereafter, the user may set the first detection unit 76 exposed from the housing 91 in the first measuring instrument 110 to construct the first sensor unit 111 and may also set the second detection unit 80 which are similarly exposed in the second measuring instrument 120 to construct the second sensor unit 121. The first sensor unit 111 and the second sensor unit 121 may be suspended from the stand 98.

The user may connect the gas-supply-channel-side connector 152b of the gas supply channel 152 extending from the standard solution storage unit 72 to the gas-supply-device-side connector 154 of the gas supply device 150. The user may also set the sample introduction channel 130 in the introduction mechanism 140 to form the introduction unit 148. Thereafter, the user may connect the sampling-device-side connector 132 of the sample introduction channel 130 exposed from the introduction unit 148 to the culturing-device-side connector 56 and may connect the plug of the sample introduction channel 130 to the connection part 84.

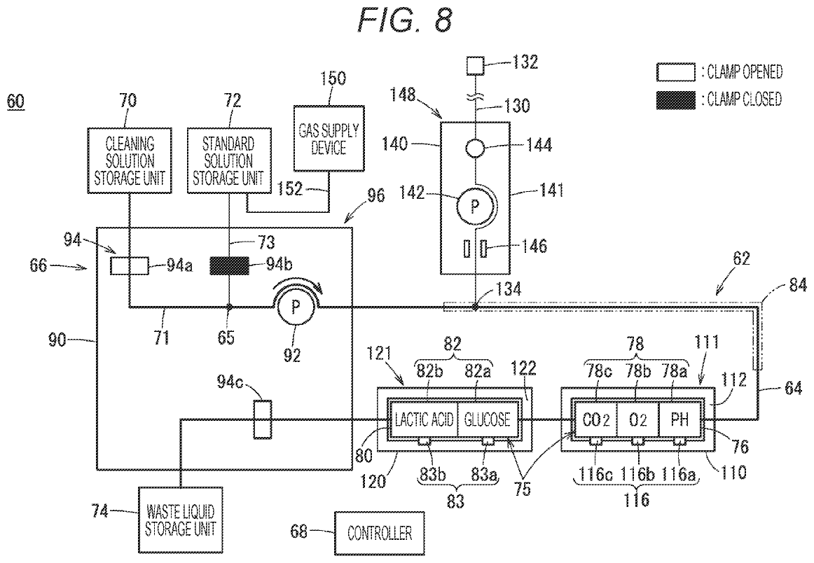

In the priming step (step S2 in FIG. 7), the controller 68 may be configured to cause the cleaning solution clamp 94a to open and also the waste liquid clamp 94c to open, to cause the standard solution clamp 94b to close, and to cause the main-mechanism-side pump 92 to rotate, for example, as illustrated in FIG. 8. Thus, the cleaning solution in the cleaning solution storage unit 70 may sequentially flow through the cleaning solution branch path 71, the branch point 65, the main-mechanism-side pump 92, the connection part 84, the first detection unit 76, and the second detection unit 80 and may be discharged to the waste liquid storage unit 74.

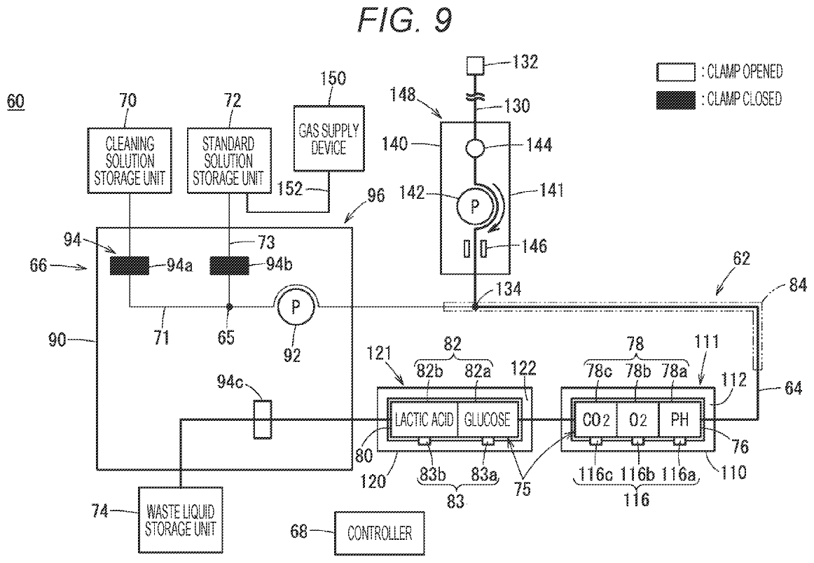

In the sampling step (step S3 in FIG. 7), the controller 68 may be configured to cause the cleaning solution clamp 94a to close and also the standard solution clamp 94b to close and to cause the waste liquid clamp 94c to open, for example, as illustrated in FIG. 9. The controller 68 may be configured to cause the introduction-mechanism-side pump 142 to rotate while stopping the rotation of the main-mechanism-side pump 92. As a result, the sample of the culturing device 11 may pass through the aseptic filter 58 (see FIG. 2) and may be introduced into the sample introduction channel 130. When flowing through the sample introduction channel 130 and passing through the introduction-mechanism-side pump 142, the sample may sequentially flow through the connection part 84 (connection point 134), the first detection unit 76, and the second detection unit 80 and may be discharged to the waste liquid storage unit 74.

When the sample passes, the plurality of first elements 78 (e.g., pH chip 78a, O₂ chip 78b, and/or CO₂ chip 78c) of the first detection unit 76 may come into contact with the sample and may be colored according to the pH and/or the contents of O₂ and/or CO₂. The first measuring instrument 110 may be configured to optically measure each of the first elements 78 and to transmit the detection result to the controller 68. The controller 68 that has received the detection result may be configured to perform appropriate processing to display the measured values (e.g., pH value, O₂ concentration, and/or CO₂ concentration) on the monitor 100.

Similarly, when the sample passes, the plurality of second elements 82 (e.g., glucose chip 82a and/or lactic acid chip 82b) of the second detection unit 80 may come into contact with the sample, and the second measuring instrument 120 may be configured to detect current values corresponding to the contents of glucose and/or lactic acid. The second measuring instrument 120 may be configured to transmit each detection result to the controller 68. The controller 68 that has received the detection result may be configured to perform appropriate processing to display the measured values (e.g., glucose concentration and/or lactic acid concentration) on the monitor 100.

After the sampling step, the controller 68 may be configured to determine whether or not the cell culture of the culturing device 11 is completed (step S4). When the cell culture is not completed (step S4: NO), the cleaning step (step S5) may be performed. In the cleaning step, the controller 68 may supply the cleaning solution in the cleaning solution storage unit 70 to the sampling channel 64, as in the priming step illustrated in FIG. 7. As a result, the sample attached to the plurality of first elements 78 (e.g., pH chip 78a, O₂ chip 78b, and/or CO₂ chip 78c) and the plurality of second elements 82 (e.g., glucose chip 82a and/or lactic acid chip 82b) may be removed by the cleaning solution.

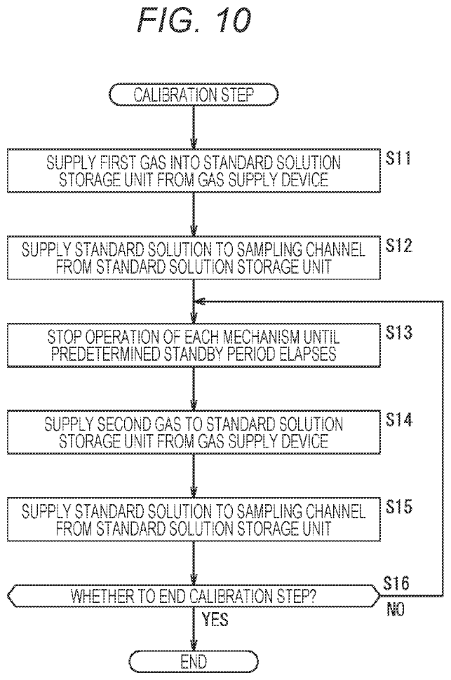

In addition, the sampling device 60 may perform a calibration step (step S6 in FIG. 7) as necessary. In the calibration step, the controller 68 may be configured to perform the calibration step according to the processing flow illustrated in FIG. 10.

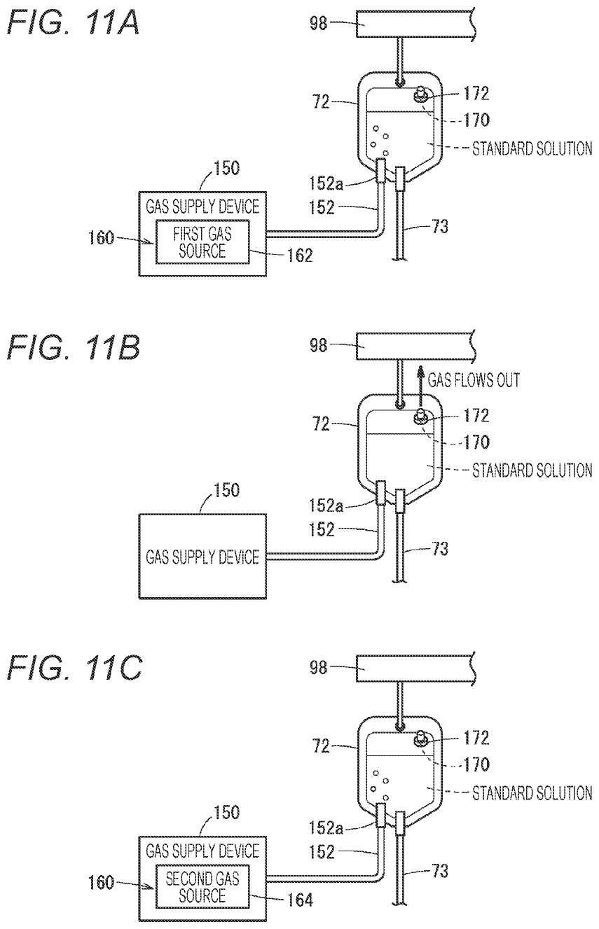

For example, the first gas may be supplied from the gas supply device 150 to the standard solution storage unit 72 (step S11) to change the component amount of gas in the standard solution to the component amount of the first gas. During this process, the controller 68 may be configured to cause all of the cleaning solution clamp 94a, the standard solution clamp 94b, and the waste liquid clamp 94c to close and to stop rotation of both the main-mechanism-side pump 92 and the introduction-mechanism-side pump 142. The controller 68 may be configured to cause the first sealing valve 163 to open and to cause the second sealing valve 165 to close to allow the first gas from the first gas source 162 to flow out of the gas supply device 150 as illustrated in FIG. 11A. The first gas may be supplied to the standard solution storage unit 72 through the gas supply channel 152 with the flow rate of the first gas being regulated by the flow rate control mechanism 166.

The controller 68 continues the supply of the first gas to the standard solution storage unit 72 for a predetermined time so that the standard solution in the standard solution storage unit 72 has an entire component amount of the first gas. After a lapse of the predetermined time, the controller 68 may be configured to cause the first sealing valve 163 to close and to cause the operation of the flow rate control mechanism 166 to stop.

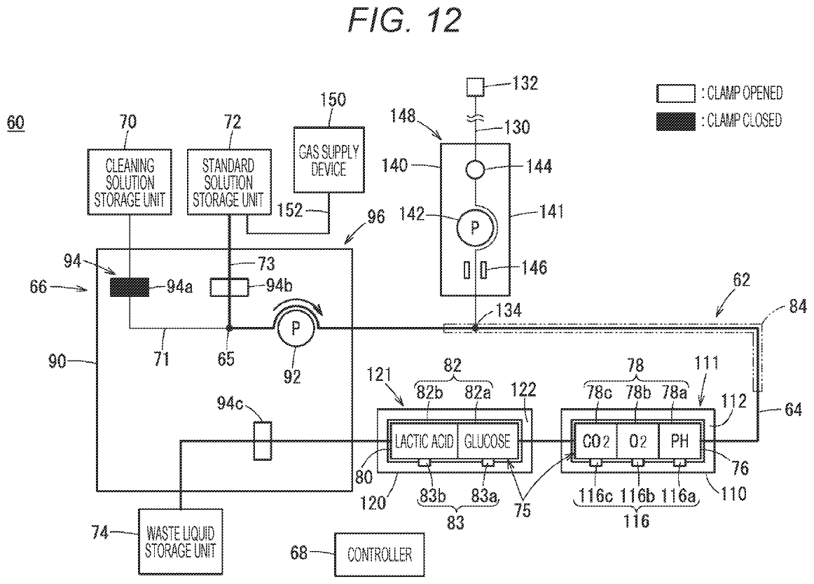

The controller 68 may be configured to supply the standard solution adjusted to the component amount of the first gas from the standard solution storage unit 72 to the sampling channel 64 (step S12 in FIG. 10) and to calibrate the first measuring instrument 110 and the second measuring instrument 120. For example, the controller 68 may be configured to rotate the main-mechanism-side pump 92 with the standard solution clamp 94b where the waste liquid clamp 94c is opened and the cleaning solution clamp 94a is closed, as illustrated in FIG. 12. Thus, the standard solution in the standard solution storage unit 72 may sequentially flow through the standard solution branch path 73, the branch point 65, the main-mechanism-side pump 92, the connection part 84, the first detection unit 76, and/or the second detection unit 80 and may be discharged to the waste liquid storage unit 74 due to the action of the main-mechanism-side pump 92.

The first sensor unit 111 (first measuring instrument 110) may be configured to measure the pH, the O₂ concentration, and the CO₂ concentration of the standard solution that has been adjusted to the component amount of the first gas and to transmit the measurement results to the controller 68 or the first measuring instrument 110. The controller 68 and/or the first measuring instrument 110 may be configured to perform calibration of each of the pH detector 116a, the O₂ detector 116b, and/or the CO₂ detector 116c (stores a first calibration point) on the basis of the measurement results. The second sensor unit 121 may be configured to measure the glucose concentration and/or the lactic acid concentration of the standard solution and to transmit the measurement results to the controller 68 or the second measuring instrument 120. The controller 68 and/or the second measuring instrument 120 may be configured to calibrate the second measuring instrument 120 on the basis of the measurement result of the second sensor unit 121.

When step S12 ends, the controller 68 may be configured to close the standard solution clamp 94b and the waste liquid clamp 94c, to stop the rotation of the main-mechanism-side pump 92, and to stop the operation of each mechanism 66 until a predetermined standby period elapses (step S13 in FIG. 10). As a result, the first gas in the standard solution storage unit 72 may be discharged to the outside of the standard solution storage unit 72 through the discharge port 170 (vent mechanism 172) during the standby period as illustrated in FIG. 11B.

The controller 68 may be configured to supply the second gas from the gas supply device 150 to the standard solution storage unit 72 (step S14 in FIG. 10) to change the component amount of gas in the standard solution to the component amount of the second gas. That is, the controller 68 may be configured to cause the second sealing valve 165 to open and to cause the first sealing valve 163 to close to allow the second gas from the second gas source 164 to flow out of the gas supply device 150 (see also FIG. 11C). The second gas may be supplied to the standard solution storage unit 72 through the gas supply channel 152 with the flow rate of the second gas being regulated by the flow rate control mechanism 166.

The controller 68 may continue the supply of the second gas to the standard solution storage unit 72 for a predetermined time so that the standard solution in the standard solution storage unit 72 has an entire component amount of the second gas. After a lapse of the predetermined time, the controller 68 may be configured to cause the second sealing valve 165 to close and to cause the operation of the flow rate control mechanism 166 to stop.

The controller 68 may be configured to supply the standard solution adjusted to the component amount of the second gas from the standard solution storage unit 72 to the sampling channel 64 (step S15 in FIG. 10) and to calibrates the first measuring instrument 110 and the second measuring instrument 120.

The first sensor unit 111 (first measuring instrument 110) may be configured to measure the pH, the O₂ concentration, and/or the CO₂ concentration of the standard solution having the component amount of the second gas and to transmit the measurement results to the controller 68 and/or the first measuring instrument 110. The controller 68 and/or the first measuring instrument 110 may perform calibration of each of the pH detector 116a, the O₂ detector 116b, and/or the CO₂ detector 116c (stores a second calibration point) on the basis of the measurement results. Then, the controller 68 and/or the first measuring instrument 110 may calibrate the gradient and the height (position) of a calibration curve indicating the relationship between the luminosity and the concentration on the basis of the stored first calibration point and second calibration point. By performing the two-point calibration in this manner, the sampling device 60 can accurately perform the calibration of the first measuring instrument 110.
the controller 68 may be configured to determine whether to end the calibration step (step S16 in FIG. 10). For example, in a case where three or more gas sources 160 are provided (step S16: NO), the processing may return to step S13 in FIG. 10 and the processing flow of steps S13 to S15 may be repeated. In other instances, when the supply of the gas from all the gas sources 160 is ended (step S16: YES), the controller 68 may be configured to end the calibration step.

With renewed reference to FIG. 7, when the cleaning step (or the calibration step) is completed, the controller 68 may be configured to returns to step S3 and to performs the subsequent steps. On the other hand, when determining in step S4 that the cell culture is completed (step S4: YES), the controller 68 may be configured to end the operation flow of the sampling device 60.

The present disclosure is not limited to the abovementioned embodiments and it should be appreciated that various modifications are possible without departing from the spirit of the invention. For example, in at least one example embodiment, the above-mentioned gas supply device 150 may be connected to the standard solution storage unit 72 via the gas supply channel 152. However, the sampling device 60 may be configured such that the standard solution storage unit 72 may be stored in the gas supply device 150 and the standard solution mixed with the first gas and the second gas in the gas supply device 150 may be supplied to the sampling channel 64. It should be appreciated that, in at least one example embodiment, that the gas supply device 150 may have a configuration in which the tank (first gas source 162 and second gas source 164) is exposed without including the housing 151.

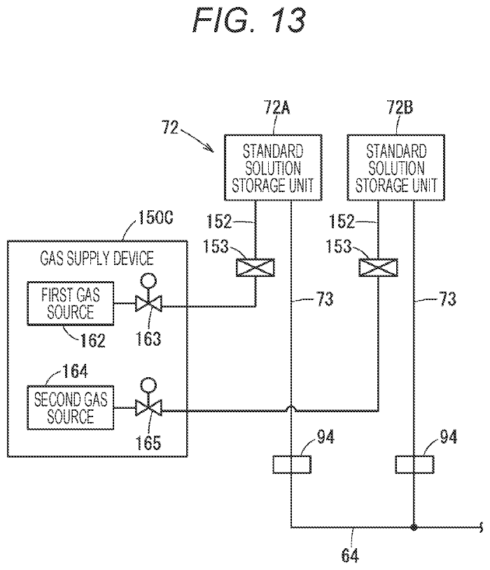

Alternatively still, as illustrated in FIG. 13, in at least one example embodiment, the sampling device 60 may have a plurality of standard solution storage units 72A and 72B, where a first gas source 162 of the gas supply device 150C may be connected to the standard solution storage unit 72A and a second gas source 164 may be connected to the standard solution storage unit 72B. With this configuration, after a standard solution in the standard solution storage unit 72A mixed with the first gas is supplied to the sampling channel 64, a standard solution in the standard solution storage unit 72B mixed with the second gas may be supplied to the sampling channel 64 without setting a standby period. Therefore, the sampling device 60 can quickly perform the calibration step.

In such instances, the standard solution in the standard solution storage unit 72A and the standard solution in the standard solution storage unit 72B may have different glucose concentrations and/or lactic acid concentrations in advance. With this configuration, two different types of standard solutions having different glucose concentrations and/or lactic acid concentrations may be supplied also to the second sensor unit 121 (see FIG. 4) and the second measuring instrument 120 may be subjected to two-point calibration.

In at least one example embodiment, the present disclosure provides a sampling device 60 for collecting a liquid sample from a cell culturing device 11. The sampling device 60 may include a sampling channel 64 through which the sample flows, a detection unit 75 provided in the sampling channel 64 so as to come into contact with the sample, and a measuring instrument (first measuring instrument 110) that measures at least a gas component contained in the sample by means of the detection unit 75 while the sample flows. The sampling device 60 may also include a standard solution storage unit 72 capable of supplying a standard solution to the detection unit 75 through the sampling channel 64 and a gas supply unit (gas supply device 150, 150A to 150C) that is configured to supply a gas having a predetermined component amount to the standard solution in the standard solution storage unit 72. The measuring instrument may perform calibration by measuring the standard solution mixed with the gas having the predetermined component amount.

With this configuration, the sampling device 60 can easily perform calibration of the measuring instrument (first measuring instrument 110) using the standard solution by supplying the gas having the predetermined component amount from the gas supply unit (gas supply device 150, 150A to 150C) to the standard solution in the standard solution storage unit 72. The sampling device 60 may also eliminate the need for a distinct calibration device, reducing the burden on the user including, for example, the need for setting the measuring instrument (first measuring instrument 110) in the calibration device.

The gas supply unit (gas supply device 150, 150A, 150C) may include a plurality of gas sources 160 that are capable of supplying gases having different component amounts to the standard solution in the standard solution storage unit 72. With this configuration, the sampling device 60 may calibrate the measuring instrument (first measuring instrument 110) at a plurality of points using a plurality of standard solutions to which gases having different component amounts are supplied, further improving the accuracy of calibration.

The gas supply unit (gas supply device 150, 150A, 150C) supplies a first gas from a first gas source 162 that is one of the plurality of gas sources 160 to the standard solution in the standard solution storage unit 72 and also supplies a second gas from a second gas source 164 that is one of the plurality of gas sources 160 at a timing different from a supply timing of the first gas. With this configuration, the sampling device 60 can supply the standard solution mixed with the first gas and the standard solution mixed with the second gas to the measuring instrument (first measuring instrument 110) at different timings.

The gas supply unit (gas supply device 150) supplies the second gas to the standard solution storage unit 72 after a predetermined standby period has elapsed after stopping the supply of the first gas to the standard solution storage unit 72. With this configuration, after using the standard solution mixed with the first gas, the sampling device 60 can supply the second gas to the standard solution in a state where the influence of the first gas is eliminated from the standard solution.

The gas supply unit (gas supply device 150A, 150B) may include a plurality of single-component gas sources 168 that stores different single gas components and that generates the gas having the predetermined component amount by adjusting a supply amount of the gas component supplied from each of the plurality of single-component gas sources 168. With this configuration, the gas supply unit may efficiently supply a target gas component to the standard solution storage unit 72.

The sampling device 60 may include a pump (main-mechanism-side pump 92) that supplies the standard solution from the standard solution storage unit 72 to the detection unit 75, and a control unit (controller 68) that controls operations of the pump and the gas supply unit (gas supply device 150, 150A to 150C). When calibrating the measuring instrument (first measuring instrument 110), the control unit may stop the pump, may supply the gas having the predetermined component amount from the gas supply unit to the standard solution storage unit 72 for a predetermined time, and may operate the pump to supply the standard solution to the detection unit 75. With this configuration, the sampling device 60 may appropriately switch between the supply of the gas to the standard solution and the supply of the standard solution to the detection unit 75 and may stably introduce the standard solution having the gas of the predetermined component amount into the detection unit 75.

The standard solution storage unit 72 includes, at one end in a longitudinal direction, an outflow channel (standard solution branch path 73) through which the standard solution may flow out to the sampling channel 64 and a gas supply channel 152 connecting the standard solution storage unit 72 and the gas supply unit (gas supply device 150, 150A to 150C) may be connected to the one end in the longitudinal direction. With this configuration, the sampling device 60 can directly mix the gas into the standard solution accumulated on the lower side in the vertical direction when supplying the gas to the standard solution storage unit 72 held such that one end in the longitudinal direction may be positioned on the lower side in the vertical direction.

The standard solution storage unit 72 may have a discharge port 170 through which the gas in the standard solution storage unit 72 may be releasable on another end side in the longitudinal direction. With this configuration, the standard solution storage unit 72 may smoothly discharge the internal gas through the discharge port 170.

The discharge port 170 may be provided with a vent mechanism 172 that allows permeation of gas and interrupts permeation of liquid. With this configuration, the standard solution storage unit 72 may prevent leakage of the standard solution to the outside when, for example, it is handled by a user.

In at least one example embodiment, the present disclosure provides a cell culturing system 10 that includes a culturing unit (culturing device 11) for culturing a cell. The cell culturing system 10 may include a sampling channel 64 through which a sample in a liquid form collected from the culturing unit flows, a detection unit 75 provided in the sampling channel 64 so as to come into contact with the sample, and a measuring instrument (first measuring instrument 110) that measures at least a gas component contained in the sample by means of the detection unit 75 while the sample flows. The cell culturing system 10 may also include a standard solution storage unit 72 capable of supplying a standard solution to the detection unit 75 through the sampling channel 64 and a gas supply unit (gas supply device 150, 150A to 150C) that supplies a gas having a predetermined component amount to the standard solution in the standard solution storage unit 72. The measuring instrument may perform calibration by measuring the standard solution mixed with the gas having the predetermined component amount. With this configuration, the cell culturing system 10 may easily perform calibration of the measuring instrument, improving usability.

1. A sampling device that receives a sample from a cell culturing device, the sampling device comprising:
a sampling channel that is configured to receive the sample;
a measuring instrument that is configured to measure one or more gas components in the sample as the sample flows through the sampling channel; and
a standard solution storage unit that is configured to supply a standard solution to the measuring instrument through the sampling channel, the standard solution having predetermined amounts of at least one selected component,
the measuring instrument being configured to perform calibration of the sampling device by measuring the standard solution.

2. The sampling device of claim 1, wherein the sampling device further includes:
a gas supply unit that is configured to supply a gas to the standard solution storage unit, the gas including a first amount the at least one selected component.

3. The sampling device of claim 2, wherein the gas supply unit includes a plurality of gas sources, the plurality of gas sources including a first gas source that is configured to store the gas in a compressed state.

4. The sampling device of claim 2, wherein the gas is a first gas, the plurality of gas sources further includes a second gas source that is configured to store the second gas in a compressed state, the second gas including a second amount of the at least one selected component, and the second amount being different from the first amount.

5. The sampling device of claim 4, wherein the gas supply unit is configured to supply the first gas and the second gas to the standard solution storage unit at different points in time.

6. The sampling device of claim 5, wherein the gas supply unit is configured to supply the second gas to the standard solution storage unit after a predetermined standby period following the supply of the first gas to the standard solution storage unit.

7. The sampling device of claim 2, wherein the at least one selected component is a first component, and the sampling device further includes:
a gas supply unit that is configured to supply a gas to the standard solution storage unit, the gas including the first component and a second component; and
the gas supply unit including a first single-component gas source that is configured to store the first component and a second single-component gas source that is configured to store the second component,
the gas supply unit being configured to generate the gas by adjusting a component amount of each of the first component from the first single-component gas source and the second component from the second single-component gas source.

8. The sampling device of claim 1, wherein the measuring instrument includes a detection unit that is disposed in the sampling channel so as to come into contact with the sample.

9. The sampling device of claim 8, wherein the sampling device further includes:
a pump that is configured to move the standard solution from the standard solution storage unit to the detection unit.

10. The sampling device of claim 9, wherein the sampling device further includes:
a gas supply unit that is configured to supply a gas to the standard solution storage unit, the gas including an amount of the at least one selected component.

11. The sampling device of claim 10, wherein the sampling device further includes:
a control unit that is configured to control operations of the pump and the gas supply unit, and when the calibration occurs, the control unit is configured to:
cause the pump to stop,
after the pump is stopped, to then cause the gas to be supplied to the standard solution storage unit for a predetermined time, and
after the gas is supplied, to then cause the pump to operate to supply the standard solution to the detection unit.

12. The sampling device of claim 11, wherein a first end of the standard solution storage unit further includes, an outflow channel through which the standard solution flows out of the standard solution storage unit and to the sampling channel.

13. The sampling device of claim 12, wherein the standard solution storage unit further includes, at the first end, a gas supply channel that connects the standard solution storage unit and the gas supply unit.

14. The sampling device according to claim 13, wherein the standard solution storage unit further includes, at a second end opposing the first end, a discharge port through which an excess gas in the standard solution storage unit is releasable.

15. The sampling device according to claim 14, wherein the discharge port includes a vent mechanism that allows permeation of the excess gas and interrupts permeation of a liquid.

16. A cell culturing system comprising:
a culturing unit for culturing a cell;
a sampling channel that is configured to receive a sample from the culturing unit;
a measuring instrument that is configured to measure a gas component in the sample as the sample flows through the sampling channel; and
a standard solution storage unit configured to supply a standard solution to the measuring instrument through the sampling channel,
the measuring instrument being configured to perform calibration of the sampling device by measuring the standard solution.

17. The cell culturing system of claim 16, wherein the measuring instrument includes a detection unit that is disposed in the sampling channel so as to come into contact with the sample.

18. The cell culturing system of claim 17, wherein the cell culturing system further includes:
a pump that is configured to move the standard solution from the standard solution storage unit to the detection unit.

19. The cell culturing system of claim 18, wherein the cell culturing system further includes:
a gas supply unit that is configured to supply a gas to the standard solution storage unit, the gas including an amount of the at least one selected component.

20. The cell culturing system of claim 19, wherein the cell culturing system further includes:
a control unit that is configured to control operations of the pump and the gas supply unit, and when the calibration occurs, the control unit is configured to:
cause the pump to stop;
after the pump is stopped, to then cause the gas to be supplied to the standard solution storage unit for a predetermined time;
and after the gas is supplied, to then cause the pump to operate to supply the standard solution to the detection unit.
